(19) United States
(12) Patent Application Publication
(10) Pub. No.: US 2007/0064309 A1
(43) Pub. Date: Mar. 22, 2007

(54) Drape Assembly For Surgical Microscope Assembly

(76) Inventors: K. Peter Luloh, Stuart, FL (US); Michael Annen, Fort Pierce, FL (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/533,690

(22) Filed: Sep. 20, 2006

Related U.S. Application Data
(60) Provisional application No. 60/719,022, filed on Sep. 21, 2005.

(51) Int. Cl. A61B 19/08

(52) U.S. Cl. 359/510; 359/507

(57) ABSTRACT

A drape assembly is provided for maintaining a sterile field around a surgical microscope. A drape has a first opening for receiving a protruding adapter connected to a lower end of the microscope and a collar is mounted in the first opening. An adhesive portion of the collar secures the drape to the adapter. The adapter becomes part of the optical system and may be referred to as an optical component.

### SPECIFIC DATA RELATED TO THE INVENTION

This application claims the benefit of U.S. Provisional application No. 60/719,022 filed Sep. 21, 2005.

### BACKGROUND OF THE INVENTION

1. Field of the Invention

The present invention relates to a surgical microscope drape assembly and, more particularly, to a drape which maintains a sterile field during the performance of a surgical procedure.

2. Background Art

Stereoscopic microscopes are used extensively in surgical environments and, in particular, during eye surgery. These microscopes are typically large and cumbersome assemblies that are maintained in operating room environments although it is impractical to sterilize these microscopes. Accordingly, various types of surgical drapes have been developed to cover non-sterilized areas of the microscope while still allowing surgeons and staff to adjust the instrumentation and use such an optical assembly without interference from the drape.

### SUMMARY OF THE INVENTION

In one form of the invention, a drape assembly is provided for maintaining a sterile field around a surgical microscope. A drape has a first opening for receiving a protruding adapter connected to a lower end of the microscope and a collar is mounted in the first opening. An adhesive portion of the collar secures the drape to the adapter. The adapter becomes part of the optical system and may be referred to as an optical component.

A method is also provided for fabricating a drape assembly of the type used to provide a sterile field around a surgical microscope. In one embodiment, the method includes providing a pliable sheet with a first opening formed in the sheet, and also providing a rigid collar with a second opening formed therein. The collar includes an area having an adhesive layer formed thereon. The collar is attached along the opening formed in the sheet to position the collar opening within the first opening and position the adhesive layer for attachment to an optical component.

A method of maintaining a sterile field around a surgical microscope is also provided. In one example the method includes providing a drape for covering the microscope, with the drape including a collar having a first opening through the drape for receiving an adapter. The collar includes a flap with a surface having an adhesive formed thereon. The adapter is attached to a lens housing of the microscope and the collar is passed along an exterior portion of the adapter so that part of the adapter extends through the collar opening. The flap of the collar is attached to the adapter or the optical component with the adhesive.

### BRIEF DESCRIPTION OF THE DRAWINGS

Features of the invention will be best understood when the following detailed description is read in conjunction with the accompanying drawings, wherein:

FIG. 1 is a perspective view of a Binocular Indirect Ophthalmo-Microscope Stereoscopic Diagonal Inverter (BIOM/SDI) system;

FIG. 2 illustrates a surgical microscope incorporating a (BIOM/SDI) system;

FIG. 3 is a plan view of a drape assembly according to the invention;

FIG. 4A is a plan view of a collar according to the invention;

FIG. 4B is a view in cross section, taken along line A-A′ of FIG. 4A, further illustrating features of the collar;

FIG. 5 is a perspective view illustrating a drape assembly; and

FIG. 6 illustrates a surgical microscope after a BIOM is attached according to the invention.

### DETAILED DESCRIPTION OF THE INVENTION

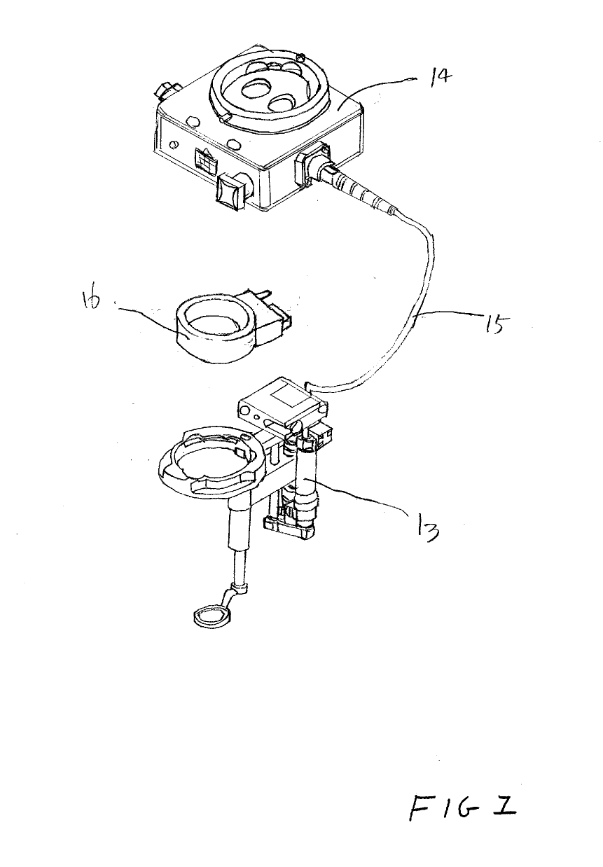

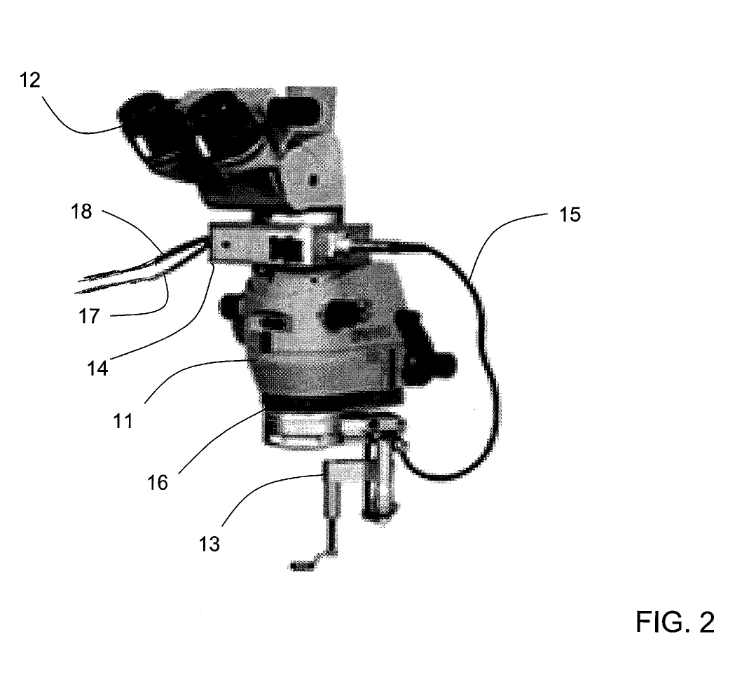

In the field of ophthalmic surgery, it is common to attach optical devices to conventional microscopes in order to further increase magnification, invert images, and facilitate stereoscopic viewing by a surgeon. One such device is a Binocular Indirect Ophthalmo-Microscope Stereoscopic Diagonal Inverter (BIOM/SDI) system. FIG. 1 illustrates a BIOM/SDI system 10 comprising a Binocular Indirect Ophthalmo-Microscope (BIOM) 13, a stereoscopic diagonal inverter (SDI) 14, a connection cable 15, and a conventional BIOM adapter 16. Normally, all of the foregoing elements, except the SDI, are sterilized before being brought into the sterilized operating room environment. FIG. 2 is a photograph of a surgical microscope 1 incorporating a BIOM/SDI system 10. The microscope 1 comprises a lens housing 11, a pair of ocular ports 12, the BIOM 13, the SDI 14, the optical cable 15 connecting the BIOM 13 and the SDI 14, the BIOM adapter 16, a power cable 17, and a switch cable 18.

According to the invention, a surgical drape fits around the microscope 1 after the adapter 16 is installed, providing an opening through which the BIOM/SDI can be attached to the microscope 1 without compromising the sterility of the surgical field. After installation of the adapter 16, the adapter may be adjusted and then tightened into a desired position on the lens housing 11 using a thumbscrew 19. Additional openings may be provided to access the ocular ports 12, and to route cabling such as the power cable 17 and the switch cable 18. The adapter 16 has a circular portion that circumscribes the lens opening of the microscope and a rectangular extension that is normally provided with a dovetail to engage a mating member on the lower end of the lens housing.

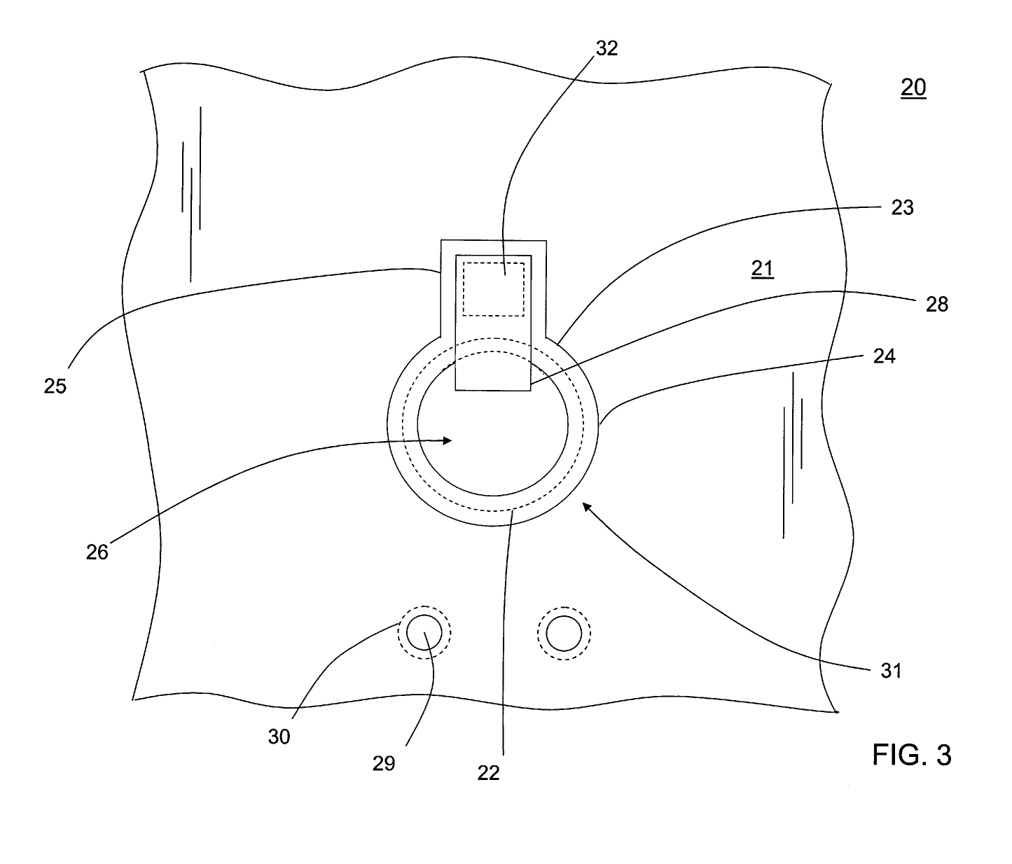

FIG. 3 illustrates an exemplary microscope drape 20 according to the invention. The drape 20 is formed of a pliable transparent plastic sheet 21 having an opening 22. The opening 22 is sized to receive a support collar 23 having of a ring portion 24 and an adjoining tab or flap 25. The ring portion 24 fits along the opening 22 and includes a ring opening 26 through which the adapter 16 may pass. The opening 26 is sized to fit around the circular portion of the adapter 16 in a manner which provides acceptable isolation between the non-sterile portion of the microscope and the portion of the adapter 16 exposed to a sterile operating room environment.

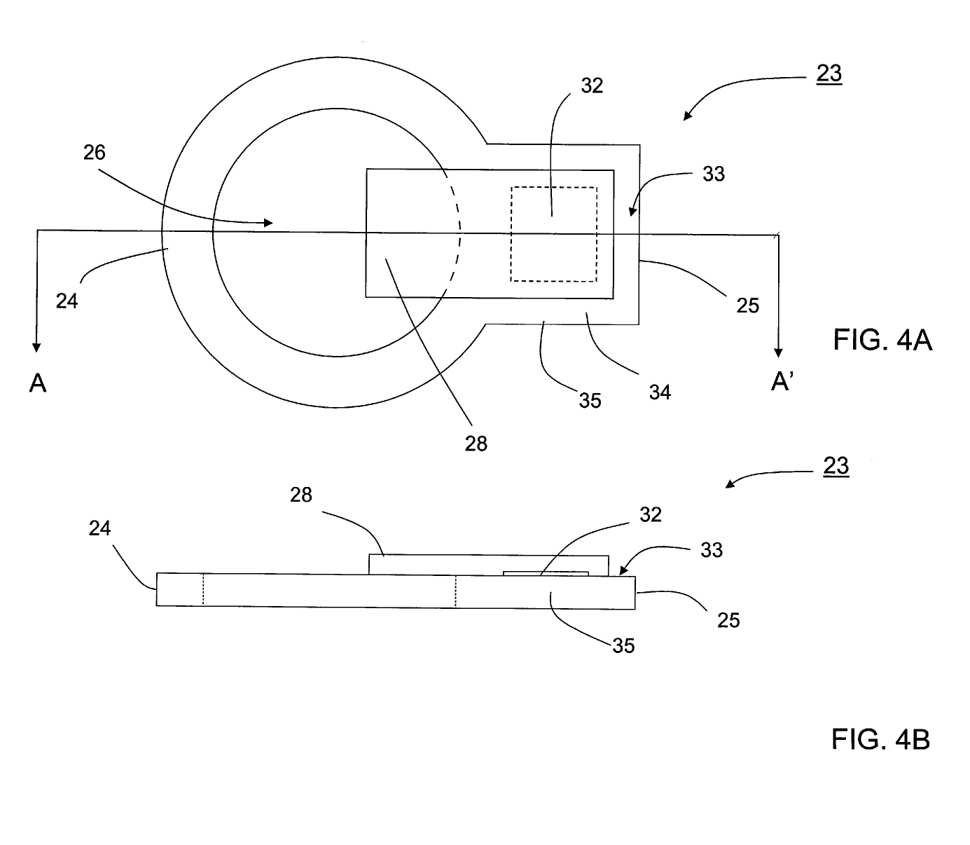

In this example, the opening 26 of the support collar 23 is concentrically aligned with the opening 22 in the drape 20. The support collar may be permanently bonded or removably attached to portions of the plastic drape 21 adjoining the opening 22 using known techniques such as an adhesive coating. With the size of the opening 26 providing a mating fit with the adapter 16 there remains sufficient clearance to allow the support collar 23 to pass about the adapter 16 and bring the flap 25 against the rectangular extension of the adapter 16. As more fully described below, the flap 25 is removably attachable to the adapter 16 extension. The support collar 23 may be produced from thin cardboard or a plastic material, e.g., 3-10 mils in thickness. When attached to the adapter 16, the collar 23 provides enough structural support to retain a portion 31 of the drape 20, in the region of the drape adjoining the opening 22, to be held about the adapter 16 or against the lens housing 11. FIGS. 4A and 4B illustrate the structure of the collar 23, comprising the ring portion 24 and the flap 25. A portion of the side 33 of the flap 25 which comes into contact with the adapter 16 is covered with an adhesive layer 32. In this example, a peripheral region 34 of the flap 25 does not have the adhesive layer 32 formed thereover. That is, the peripheral region 34 provides a non-stick area along an edge 35 of the flap 25 which can be easily grasped for removal of the collar from the lens housing 11 and adapter 16.

Prior to use of the microscope drape 20, the entire adhesive layer 32, or even the entire side 33, is covered with a removable cover, e.g. peel-off paper or plastic cover 28. With the cover 28 installed, the adhesive layer 32 may be protected from the environment prior to use of the drape 20 but can be quickly deployed by removal of the cover.

The drape 20 can be designed to fit a wide variety of microscopes. The support collar 23 may be of a design allowing for its use in different sized drapes and drape openings and with adapters or other devices of varying size. That is, the ring portion 24 of the collar 23 can be formed of a material or a design which allows the opening 26 to be expanded. For example, the opening 26 may be formed in a cardboard collar with radial slits allowing portions of the cardboard along the opening to be displaced as an adapter of slightly larger diameter than the opening is passed through the opening. Alternately, the ring portion 24 may be formed of a deformable or elastic material allowing the opening 24 to be stretched to a larger size. The drape 20 has additional openings 29 at various locations, e.g., to pass eye pieces, cables or cords, of the microscope through the drape 20. Such openings may be formed with peel-off covers 30 to retain a barrier between the sterile environment and the non-sterile region within the drape when the openings are not required to provide a function.

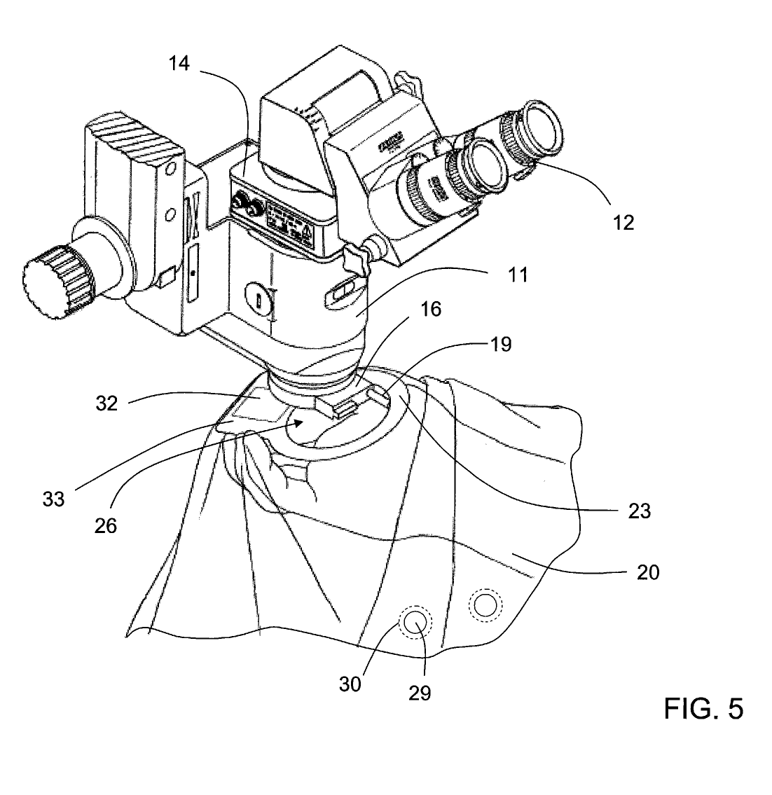

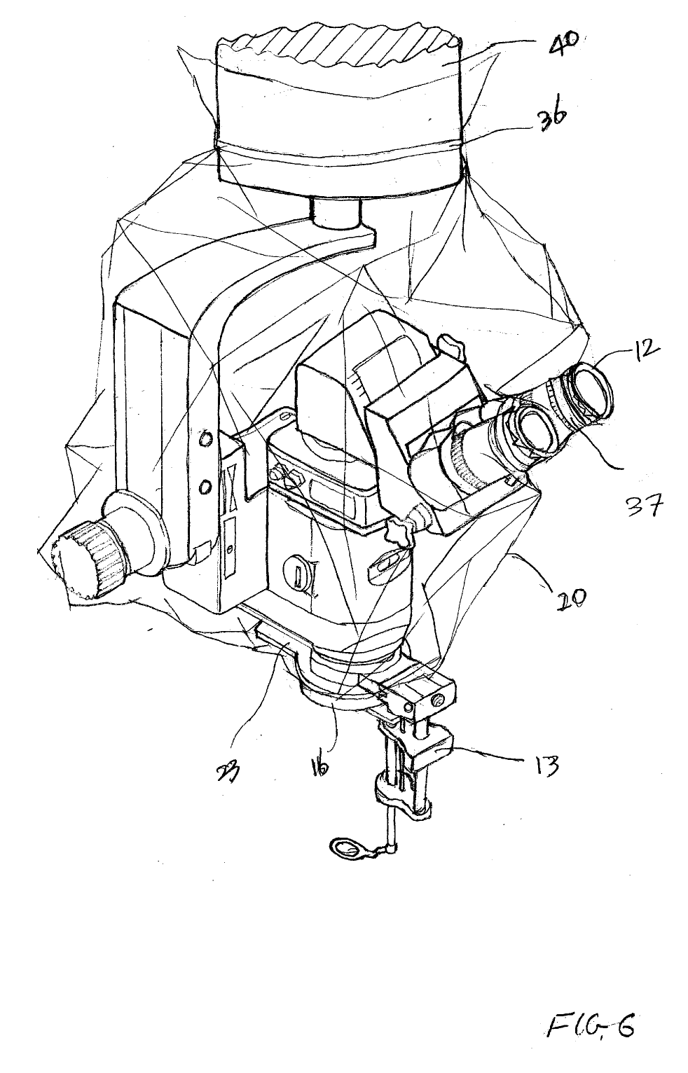

FIG. 5 illustrates the surgical drape 20 positioned at an initial stage of installation on the microscope 1 with the adapter 16 (which may have been sterilized) having already been installed on the lens housing 11. At this stage the adapter 16 may be tightened into position on the lens housing 11 using the thumbscrew 19 provided on the adapter, although readjustment may be desired after the drape 20 is installed. With the adapter 16 positioned on the lens housing, in a method of maintaining a sterile field around the surgical microscope 1, the support collar 23 is passed along the adapter 16 such that the adapter protrudes through the collar opening 26 and the side 33 of the flap 25 faces the extension of the adapter 16. The cover 27 is peeled off of the adhesive layer 32 on the side 33 of the flap 25. The flap 25 is then pressed upward and against a surface of the adapter 16 extension. This action secures the portion 31 of the drape 20 so that it does not fall downward along the adapter and over the ocular attachments that are to be connected to the adapter 16, e.g., the BIOM 13. As illustrated in FIG. 6, the drape is positioned to cover the microscope 1 and is wrapped against the microscope 1 and a post 40 to which the microscope is attached by tying a sterile tie strap 36 thereabout. The adapter 16 provides a sterile attachment point for additional optics to be used during ophthalmic surgery. With this arrangement the sterile BIOM 13, also shown in FIG. 6, is attached to the adapter 16, allowing surgical staff to proceed to work in a sterile field. When additional openings with peelable covers are provided, e.g., to access the ocular ports 12 and openings for cables, the openings are positioned to receive these optical and electrical elements and the covers are removed accordingly. FIG. 6 shows the ocular ports 12 tied with sterile straps 37 after the covers are removed.

While one embodiment of the invention has been illustrated and described, the invention is not so limited. Many variations or improvements can be made without departing from the spirit and scope of the invention which is only limited by the claims which follow.

We claim:

1. A drape assembly for maintaining a sterile field around a surgical microscope of the type having a lens housing, comprising:
a drape having a first opening for receiving a protruding adapter connected to the lens housing; and
a collar mounted in the first opening with an adhesive portion for securing a portion of the drape to the adapter.

2. The drape assembly of claim 1 including a plurality of additional openings for receiving one or more ocular ports or electrical connectors associated with the microscope.

3. The drape assembly of claim 1 wherein the collar is formed of cardboard or a plastic sheet.

4. The assembly of claim 2 wherein some of the additional openings in the drape assembly are adapted to receive detachable covers.

5. A method of fabricating a drape assembly of the type used to provide a sterile field around a surgical microscope having a lens housing, comprising:
providing a pliable sheet with a first opening formed in the sheet;
providing a collar with a second opening formed therein and an area having an adhesive layer formed thereon;
attaching the collar about the first opening so that a sterile component may pass through the first opening and the second opening; and
positioning the adhesive layer for attachment to an optical component.

6. The method of claim 5 wherein the second opening has a diameter permitting a component mounted to the microscope to pass therethrough.

7. The method of claim 5 further comprising the step of providing additional openings capable of receiving detachable covers.

8. The method of claim 7 wherein the additional openings are positioned to provide access to one or more ocular ports or electrical ports of the microscope.

9. A method of maintaining a sterile field around a surgical microscope, comprising the steps of:
providing a drape for covering the microscope, the drape including a collar having an opening extending through the drape for receiving an optical component and a flap including a surface area having an adhesive formed thereon;
attaching the optical component to a lens housing of the microscope;
passing the collar opening about an exterior portion of the optical component so that part of the optical component extends through the collar opening; and
adhesively attaching the flap of the collar to the optical component associated with the microscope the adhesive to secure a portion of the drape about the collar.

10. The method of claim 9 wherein the step of providing the drape includes providing a plurality of additional openings in the drape and the adhesive secures the portion of the drape to a lens housing.

11. The method of claim 9 further including wrapping the microscope with the drape by tying a sterile tie strap around the microscope.
